(19) United States
(12) Patent Application Publication
(10) Pub. No.: US 2024/0009024 A1
(43) Pub. Date: Jan. 11, 2024

(54) A MARKED MONOFILAMENT RETRIEVAL THREAD FOR USE WITH INTRAUTERINE SYSTEMS OR DEVICES OR OTHER INTRACAVITARY DEVICES AND THE METHOD OF MAKING

(71) Applicant: Rohini Sakuntala Seneviratne, Nawala (LK)

(72) Inventor: Rohini Sakuntala Seneviratne, Nawala (LK)

(21) Appl. No.: 18/020,935

(22) PCT Filed: Dec. 28, 2021
(86) PCT No.: PCT/IB2021/062363

§ 371 Date: Feb. 12, 2023

(30) Foreign Application Priority Data
Jan. 1, 2021 (LK) 21536

(51) Int. Cl. A61F 6/18; A61F 6/14

(52) U.S. Cl. CPC A61F 6/18; A61F 6/144

(57) ABSTRACT

A monofilament retrieval thread for use with intrauterine systems/devices or other indwelling intracavitary devices, providing a manufactured intrauterine system/device having a retrieval thread with one or more tails of approximately equal length marked with gradations at fixed uniform intervals which are inert to biological fluids, are sono- and radio-opaque. The device is positioned inside the uterus of a woman seeking gynaecological intervention or positioned inside a body cavity for diagnostic or therapeutic purposes.

### 1. FIELD OF THE INVENTION

The invention relates to a retrieval thread or retrieval string for use as a component of, or attachment to, a frame of an intrauterine system or device, or other device used in a clinical setting inside the human body. Specifically, it relates to a monofilament thread or string, with a uniform, fixed, graded, scale marked on it at manufacture with an inert material that is sono- and radio-opaque and visible to the naked eye, consisting of one or more ‘tails’ side by side, of approximately equal length.

### 2. BACKGROUND ART

An intrauterine system or device (IUS/IUD) is a small plastic frame with an attached thread that is inserted into the womb, or uterus, by a trained medical practitioner, to achieve reversible contraception, for the relief of menstrual dysfunction, for providing the patency of the uterine cavity, or rarely for localising a misplaced previously inserted device.

IUS/IUDs vary in shape and size and may have agents such as hormones or copper added, for specific objectives. The thread on the system is for its retrieval and consists of two ‘tails’ side by side. At present there are no markings provided on the thread.

After womb (uterine) size and position are assessed by inspection, through the vagina using an instrument called a speculum, the length of the uterine cavity is ascertained, and the IUS/IUD to be used is decided. Currently the ‘accepted’ range of cavity length is 6 to 15 cm.

The IUS/IUD is then fitted using an inserter/introducer, usually a plastic tube-like structure with a piston mechanism. The correctly fitted IUS/IUD is near the farther limit, the fundus of the uterine cavity (see FIG. 2). The inserter is withdrawn, the tails of the retrieval thread protrude from the external opening or external os (os herein), of the uterine neck, or cervix, and the thread is trimmed so that approximately 2 to 3 cm extend out.

In a relatively new practice, the IUD is inserted immediately after parturition (giving birth), whether by caesarean section or by vaginal birth. The uterus is larger at this time (the distance ‘Y’ shown in FIG. 2 is longer) and a longer inserter, and a longer thread, are used (as shown in FIG. 3). At caesarean the IUD is placed manually under direct vision at the uterine fundus, and at vaginal birth inserted vaginally and through the uterine cervix. The thread in both cases protrudes out through the cervical os. Thread trimming is done at an interval after involution of the uterus.

The woman is usually taught to self-examine and feel the threads of the IUS/IUD to confirm that it is in place. A follow-up visit is sometimes required to confirm position, by subjective visual estimation, aided by a speculum. Thread trimming is performed again at this visit if the insertion was immediate after parturition.

The IUS/IUD can be retained, for varying durations, depending on the clinical requirement. In the currently used devices, the duration can vary from a few weeks up to ten (10) years, depending on the purpose of insertion. The position of the IUS/IUD may need to be determined multiple times over such durations, if the wearer cannot feel the threads, feels too much thread protruding or feels part of the device in the upper part of the vagina. In instances which the wearer has not come in for removal post-insertion, at the stipulated time this duration may be longer. Devices have a pre-insertion shelf life of about five (5) years and up to ten (10) years post-insertion, depending on the device.

The frame and thread of the IUS/IUD must be inert to body fluids and other fluids with which they come into contact. These are the fluids within the uterine cavity at various stages of the menstrual cycle, vaginal discharge both physiological and pathological, seminal fluid, lubricant gels and spermicidals used during coitus, and the fluids of the peritoneal cavity. The usual vaginal pH is between 3.8 and 5.0, which is moderately acidic. Also, during diseases this goes up. The gastric pH is 1.5 to 3.5. Pancreatic pH is about 7.5-8.

The IUS/IUD is a low-cost and commonly used method of long-acting reversible contraception (LARC). Globally it has high levels of patient acceptance, a comparatively good safety profile, and data for efficacy and side effects are available from over thirty years of post-marketing surveillance.

Other medical devices are used within other parts of the body for various therapeutic and diagnostic purposes such as administering of medications, delivering electrical currents and establishing patency of blocked tubes and vessels. They are inserted and immediately withdrawn or indwelling for varying lengths of time. Some of these are marked and the marking may or may not have gradations. An example is the epidural catheter Portex®/Perifix®, where the markings aid positioning, drug delivery and localizing using direct vision and imaging techniques.

### 3. TECHNICAL PROBLEM

There are several drawbacks in the existing technology relating to IUS/IUDs.
a. While or after the inserter of an IUS/IUD is withdrawn the frame of the IUS/IUD may be displaced. Assessing its position is important. Malpositioned IUSs can lead to complications such as expulsion, displacement, embedding or impaction in the uterine wall, and perforation. These situations require management in different ways.
b. With the relatively recent technique of inserting an IUD immediately after childbirth (postpartum insertion), there is a further need to refine/ensure correct placement and enable objective assessment of displacement or migration farther upward, of the device.
c. In certain situations, the end of the retrieval thread disappears upward through the os and then the thread cannot be detected by touch or beyond a certain distance even by sight. The “missing thread” can lead to anxiety and fear in the patient.
d. Presently, a thread which is out of sight cannot be detected by remote imaging. If and when its end can be seen, whether protruding out of the os or not, it is not possible or very difficult to determine the location of an IUS/IUD with sufficient precision.
e. This uncertainty can lead to inappropriate or over-intervention in the repositioning or removal of a displaced device.
f. The problems described above also hinder patient acceptance further of what is a relatively safe method of LARC.

### 4. TECHNICAL SOLUTION

The proposed invention which contains a marked monofilament thread as a retrieval thread for use with IUS/IUDs and other indwelling devices within the relevant body cavities addresses the technical problems mentioned above.

The proposed monofilament thread contains marked gradations at uniform and fixed intervals, which are radio—and sono—opaque and visible to the naked eye, and in a material that is inert to biological fluids in the intrauterine and vaginal environment, the abdominal cavity and other cavities of the human body.

The frame part of most commonly used IUS/IUDs is radio- and sono-opaque. The addition of the proposed markings to the retrieval thread makes it possible to confirm easily and quickly the correct placement and if displaced, then to define the path and extent of the displacement.

The proposed invention aims to:
a. Enable objective assessment of correct placement of the IUS/IUD by direct observation of the retrieval thread.
b. Enable correct assessment of the length of the retrieval thread of the IUS/IUD, more accurate thread trimming, and more precise documentation at thread trimming.
c. Localize the retrieval thread by remote imaging and thereby ensure correct placement, and assessment of displacement and/or migration, of the IUS, in instances where none, too little or too much of the thread can be visualized directly, immediately on post-insertion or subsequently.
d. Confirm the integrity of the knot of the retrieval thread at the frame (for ensuring secure attachment) by remote imaging.
e. Minimize inappropriate or over-intervention in the removal of a displaced IUS/IUD.

### BRIEF DESCRIPTION OF THE DRAWINGS

FIG. 1: An embodiment of a retrieval thread with proposed markings

FIG. 2: A retrieval thread with proposed markings shown attached to an intrauterine device placed in the uterine cavity

FIG. 3: Currently used CuT 380 postpartum inserter and standard inserter with IUD

### 6. SUMMARY OF THE INVENTION

The present invention discloses a monofilament retrieval thread for use with intracavitary devices which is comprised of one or more tails having gradation markings at uniform intervals. The thread consists of two contrasting colours, alternatingly, one of the colours forming gradation markings that are radio—and sono—opaque and visible to the naked eye. The thread and the markings are resistant to degradation by both acids and biological fluids of the human body at a pH range of 1-9. The thread is formed by extruding a melt stream of polymeric material in the said colours.

The two contrasting colours of the thread are formed by uncoloured and coloured polymer extruded alternatingly at specific calculated volumes of polymeric material at specific calculated intervals during the process of manufacture.

A 1 mm long gradation marking is made at each uniform interval on the thread and additional 1 mm long gradation markings are made at specified intervals for convenient identification of the length at each such specific marked interval.

The thread is manufactured by extruding a melt stream of polymeric material periodically alternated between two contrasting colours at specific calculated volumes of polymeric material at specific calculated intervals and extruding a melt stream of polyethylene material forming a continuously coloured 22-25 mm long section at the midpoint of the thread, used for knotting to the proximal end of the frame of the intracavitary device. The alternation between two contrasting colours form a pattern of a measuring scale having gradation markings at uniform intervals, illustrating the length of the thread. The gradation markings are both radio- and sono-opaque and visible to the naked eye.

The two contrasting colours on the thread are achieved by uncoloured and coloured polymer extruded alternatingly.

The pattern or measuring scale having gradation markings on the thread comprises a 1 mm long gradation marking made at uniform intervals on the thread and additional 1 mm long gradation markings made at specified intervals for convenient identification of the length at each such specific interval resulting in a measuring scale marked along the entire length of the thread.

### 7. ADVANTAGEOUS EFFECTS

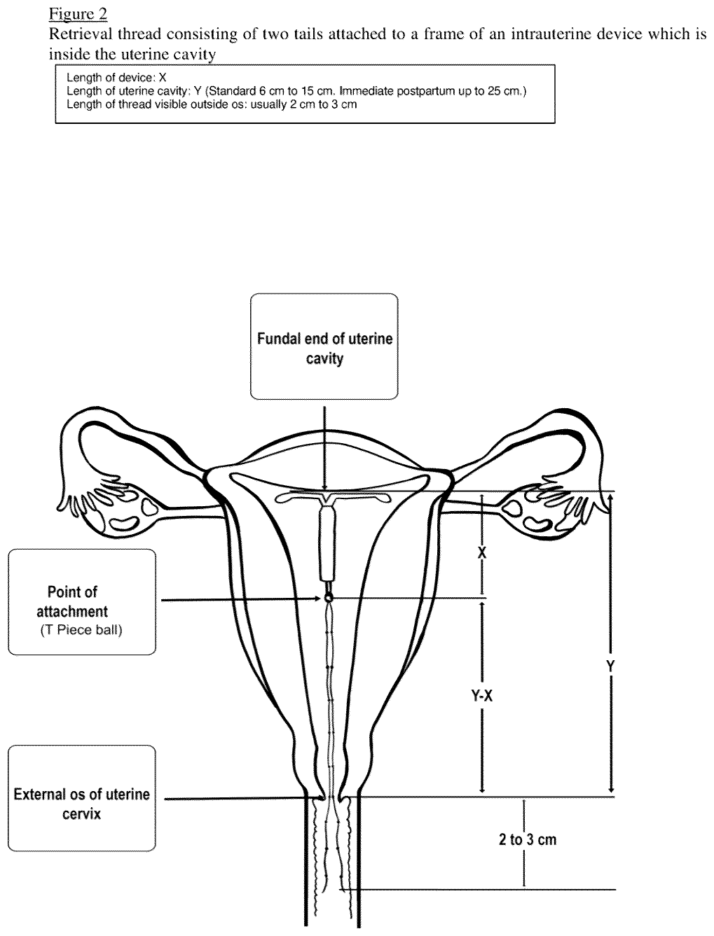

1. a) Under prior art, after inserting the IUS/IUD and withdrawing the inserter/introducer, accurate placement of the IUS/IUD can only be assumed as corresponding to the cavity length as previously measured (by an instrument known as the uterine sound). The depth of placement (Y in FIG. 2) is known by the markings on the inserter/introducer, as noted at insertion only, with no comparable markings on the device or retrieval thread. There is no method to detect the position of the frame based on the length of thread protruding at the os. The displacement of the frame on withdrawal (e.g., due to the inserter/introducer then being shaken sideways) cannot be ascertained immediately and an assumption, which may be erroneous, is made that the frame has remained static.
b) With the proposed invention the presence of the markings on the thread will permit objective assessment of the position of the frame. The length of thread denoted by the visualised mark closest to the os plus frame length correspond to cavity length (Y in FIG. 2) if placement is accurate.
2. a) Under prior art, if device displacement at insertion is suspected, recourse to ultrasound equipment is necessary to confirm clinical suspicion. This may not be available in low-resource settings.
b) With the proposed invention, as objective thread length corresponds to depth of placement the need for immediate ultrasonic assessment to rule out dislodging is much less.
3. a) Under prior art, the retrieval thread is trimmed using a visual estimation and not an objective indication of length, because the thread has no visible gauge/scale markers.
b) With the proposed invention the presence of markers permits objective measurement for trimming.
4. a) Under prior art, documentation of an objective measure cannot therefore be achieved. Post-insertion checks for displacement be it migration or expulsion, of the IUS/IUD may thus be inaccurate.
b) With the proposed invention, the presence of the markers enables objective documentation and post-insertion checks for placement are accordingly more accurate.
5. a) Under prior art, in the event of migration or incomplete expulsion remote imaging such as ultrasound or x-ray cannot be used to locate the threads as they cannot be observed by such methods and only the site of the frame of the system can be detected. Lack of information on the position of the thread results in difficulty in retrieval, and in deciding whether surgery or a procedure which avoids surgery is needed.
The procedure decided on can be retrieval in an outpatient setting, carried out in the clinic, as a minimally invasive low-risk intervention using an atraumatic retriever, or artery forceps, as compared with an in—hospital stay for higher—risk procedures such as hysteroscopy, laparoscopy or laparotomy.
b) With the proposed invention the thread can be seen using ultrasound or x-ray. The more correct information thus made available will facilitate more appropriate patient counselling, to decide on the necessity for surgery, more informed planning of surgery, or the setting for the retrieval procedure. It will prevent over-intervention, and also avoid unsuccessful and painful outpatient attempts at retrieval.
6. a) Under prior art, when insertion is immediate postpartum (within 10 min. up to 48 hrs.) then with subsequent involution of the uterus to an expected average cavity length a longer length of the thread than at the time of insertion is expected to be seen protruding at the os on the 6-week postpartum follow-up visit. This assumes there has been no upward migration or downward displacement of the frame of the device, and considers only the physiological process of involution, with the relative downward movement of both uterus and cervix together with the contained device, causing thread extrusion. At the follow-up visit the thread is trimmed at 2-3 cm protrusion length by visual estimation.
An objectively measured deviation from the average uterine placement depth expected can indicate expulsion or migration, but this cannot presently be achieved by visual estimation of the unmarked protruding thread.
b) With the proposed invention, the presence of the markers enables correct assessment of placement, and objectively measured trimming, as opposed to estimated trimming, at the stipulated length.
The ability to clinically estimate uterine size with palpation (physical examination by pressure of the fingers on the surface of the body) and corelating this (smaller uterus, more thread protruding; larger uterus, less thread) to the trimmed thread length also facilitates the estimating of correct siting. This is relevant in a low-resource setting with no recourse to ultrasound, and in which, also, postpartum insertions are predicted to be more frequent.
7. a) Under prior art, the lack of a visible marker or markers makes it not possible to document an objective measurement to be used for “interval thread trimming” (thread trimming at the follow-up visit after insertion). This hinders later follow-up checks for displacement, be it migration or expulsion.
b) With the proposed invention the visible markers enable objective measurement for trimming. Follow-up outpatient visual checks will thus be objective assessments, and objectively guide the decision on whether imaging is necessary or not.
8. a) Under prior art, the detachment of the unmarked retrieval thread from the frame cannot be identified by remote imaging. Often this is unsuspected where the thread is visible at the cervix uteri and leads to false reassurance.
b) The proposed invention of the marked segment at the point of attachment of the thread to the frame allows ultrasonic and x-ray detection of the integrity of the knot there.

### 8. MODE OF INVENTION

The invention is intended to replace the unmarked retrieval thread presently used with IUS/IUDs, and other intracavitary devices requiring retrieval. The proposed solution is the provision of graduated measuring scale markings, which are inert to biological fluids in the intrauterine and vaginal environment, the abdominal cavity, and the other cavities of the human body, providing fluoroscopic, ultrasound, and x-ray visibility, and visible to the unaided eye, placed at fixed, uniform intervals on the retrieval thread used with IUS/IUDs and with other devices that are inserted into and need retrieval from a body cavity.

In an embodiment, the proposed invention consists of a monofilament thread, for the IUS/IUD or other device, that will form one or more tails side by side subsequent to attachment, which contains markings that are visible fluoroscopically, are radio—and sono—opaque, and also visible to the unaided eye.

Material used for the thread (string) will comply with the specifications as per the World Health Organisation (WHO)/United Nations Family Planning Association (UNFPA) guidelines as set out at the end of this document as Annexure 1.

In an embodiment the monofilament is formed of a high-density polyethylene (HDPE) which is free of stabilizers.

Some suggested and selected specifications for the material used for the thread or string are as follows:

| | |
|---|---|
| Example of suggested dye | FosterLoPro |
| Thread diameter | 0.25 ± 0.05 mm |
| Shelf life | 5-7 years |
| Ph range at which the material | 4.2-7.94 (for intrauterine devices/systems) |
| of the thread and marks are | 1-9 (for gastrointestinal devices/systems) |
| resistant to degradation | |
| In situ time | About 12 years |

In a preferred embodiment the appearance of the thread consists of two contrasting colours alternating at uniform intervals forming markers along a graduated scale.

In a preferred manufacture of this embodiment the melt stream of polymeric material being extruded as the monofilament thread is periodically alternated between polymeric material of two contrasting colours, (or uncoloured and coloured polymer), at specific calculated volumes of material at specific calculated intervals, resulting in the desired appearance of background and markers in the finished monofilament. The process of polymer extrusion can be done through conventional methods or the use of three-dimensional printing using available technology.

The intervals for the gradations/markers can be centimetres, millimetres, or inches, or whole, fractional, or other multiples of these or other units.

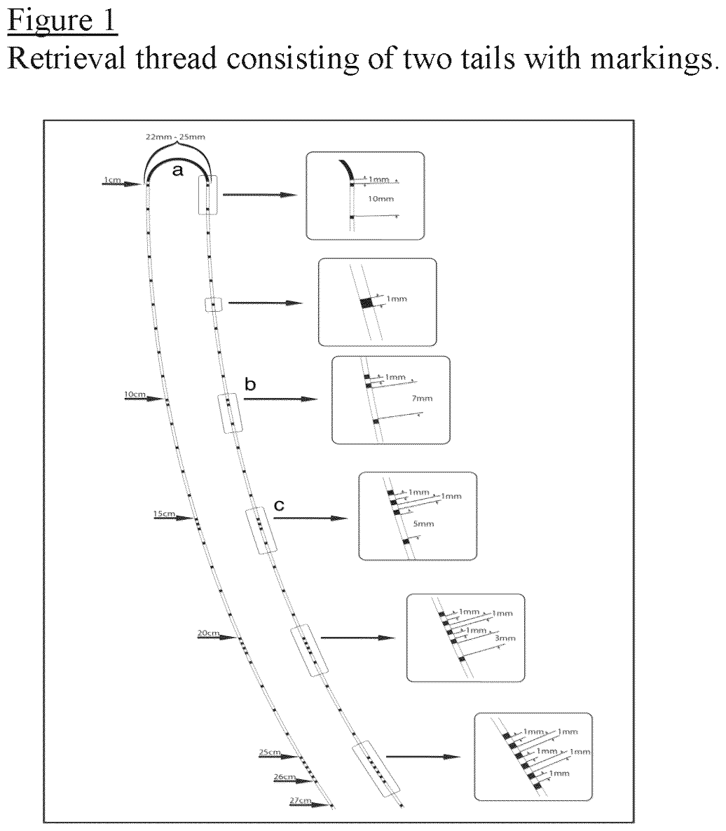

The invention proposes that one or more tails of the monofilament retrieval thread be marked at 1 cm or other gradations at fixed uniform intervals (the example shown in FIG. 1 is for two tails), using materials as described above, to enable objective assessment of displacement and/or migration of the device described and/or its thread by remote imaging and direct visualising (see FIG. 1).

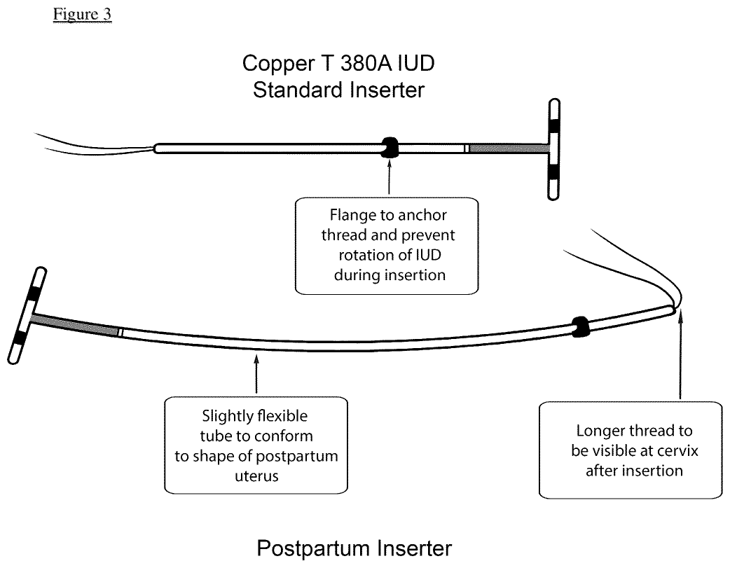

The thread is to be made in two sizes such that the longer thread length facilitates use with the postpartum inserters where the cavity length and introducer are longer (FIG. 3). It will be provided in two lengths of about, but not limited to, 3600 mm and 5400 mm, with a diameter 0.25±0.05 mm.

In a preferred embodiment the proposed thread is knotted to the frame at the continuously coloured 22-25 mm long section at its midpoint (a in FIG. 1). The knot represents the 0 mark, and the adjacent continuously coloured length the first 1 cm of each of the two tails of the retrieval thread. The accuracy of the scale will be ±*x* mm depending on the knot. This is adequate for its purpose.

The mark clusters on the retrieval thread are first at 10 cm (b in FIG. 1), and thereafter at multiples of 5 cm, the number of marks at each cluster representing that multiple, e.g., 3 marks at 15 cm (c in FIG. 1). This facilitates counting up or down for objective measurement.

The following is an illustrative example where the retrieval thread is attached to the proximal end (the end nearer the observer, away from the fundus) of the frame:

The average IUS/IUD frame is 3 to 3.5 cm in length. The average cavity length is 7-8 cm. 2-3 cm is the desired length of thread protruding at the os. For an 8 cm cavity with a 3 cm device correctly inserted, the length of thread within will be 5 cm, and the protrusion will be 2-3 cm. Example of documentation post-insertion for an 8 cm cavity with a 3 cm device would read, “Thread trimmed at 7 cm with 2 cm at the os”.

Where the retrieval thread is knotted to the farther end of the frame (with or without over molding) the measured cavity length will equal the intracavitary length of the thread.

Described below is an embodiment, where the method of markings for the longer thread (for the postpartum requirement as described) consisting of two tails uses a centimetre scale, proceeding from one end of it to the midpoint and then to the other end (FIG. 1). The shorter thread is truncated at about the mark denoting 18 cm on both halves of the thread but is otherwise identical to the longer thread.
1. The markings commence after 9 mm of unmarked thread.
2. This is followed by a coloured band of 1 mm thickness (denoting 27 cm from the attachment point).
3. Followed by an interval of 9 mm of unmarked thread
4. Followed by 6 coloured bands of 1 mm thickness with intervals of 1 mm of unmarked thread between consecutive bands (the first band denoting 26 cm, the cluster of second to sixth collectively denoting 25 cm, and the sixth at 25 cm)
5. Followed by an interval of 9 mm of unmarked thread
6. Followed by 4 coloured bands of 1 mm thickness with intervals of 9 mm of unmarked thread between consecutive bands (the bands denoting 24, 23, 22, and 21 cm)
7. Followed by an interval of 3 mm of unmarked thread
8. Followed by 4 coloured bands of 1 mm thickness with 1 mm of unmarked thread between consecutive bands (collectively denoting 20 cm)
9. Followed by an interval of 9 mm of unmarked thread
10. Followed by 4 coloured bands of 1 mm thickness with intervals of 9 mm of unmarked thread between consecutive bands (the bands denoting 19, 18, 17, and 16 cm)
11. Followed by an interval of 5 mm of unmarked thread
12. Followed by 3 coloured bands of 1 mm thickness with 1 mm of unmarked thread between consecutive bands (collectively denoting 15 cm)
13. Followed by an interval of 9 mm of unmarked thread
14. Followed by 4 coloured bands of 1 mm thickness with intervals of 9 mm of unmarked thread between consecutive bands (the bands denoting 14, 13, 12, and 11 cm)
15. Followed by an interval of 7 mm of unmarked thread
16. Followed by 2 coloured bands of 1 mm thickness with 1 mm of unmarked thread between them (collectively denoting 10 cm)
17. Followed by an interval 9 mm of unmarked thread
18. Followed by 9 coloured bands of 1 mm thickness with intervals of 9 mm of unmarked thread between consecutive bands (the bands denoting 9 cm to 1 cm)
19. Followed by an interval of 1 mm unmarked thread
20. Followed by around 20 -23 mm of continuously coloured thread

The length of 20 to 23 mm of continuously coloured thread and the two 1 mm unmarked gaps on either side of it denote the 0 to 1 cm lengths, once the thread is knotted to form the two tails. It leaves some excess for secure knotting, approximately 5 mm, to attach to the T-piece ball (or other attachment point) of the frame (See FIG. 2). As stated previously, accuracy to the last mm is not required as thread trimming is done at 2 to 3 cm of the length protruding at the os. The accuracy of the scale will be ±*x* mm depending on the knot or length of thread used for attachment, and this is adequate for its purpose.

(The markings follow from here as given below in reverse order to the above.)
1. Followed by an interval 1 mm of unmarked thread
2. Followed by 9 coloured bands of 1 mm thickness with intervals of 9 mm of unmarked thread between consecutive bands (the bands denoting 1 cm to 9 cm)
3. Followed by an interval of 9 mm of unmarked thread
4. Followed by 2 coloured bands of 1 mm thickness with 1 mm of unmarked thread between them (collectively denoting 10 cm)
5. Followed by an interval of 7 mm of unmarked thread
6. Followed by 4 coloured bands of 1 mm thickness with intervals of 9 mm of unmarked thread between consecutive bands (the bands denoting 11, 12, 13, and 14 cm)
7. Followed by an interval of 9 mm of unmarked thread
8. Followed by 3 coloured bands of 1 mm thickness with 1 mm of unmarked thread between consecutive bands (collectively denoting 15 cm)
9. Followed by an interval of 5 mm of unmarked thread
10. Followed by 4 coloured bands of 1 mm thickness with intervals of 9 mm of unmarked thread between consecutive bands (the bands denoting 16, 17, 18, and 19 cm)
11. Followed by an interval of 9 mm of unmarked thread
12. Followed by 4 coloured bands of 1 mm thickness with 1 mm of unmarked thread between consecutive bands (collectively denoting 20 cm)
13. Followed by an interval of 3 mm of unmarked thread
14. Followed by 4 coloured bands of 1 mm thickness with intervals of 9 mm of unmarked thread between consecutive bands (the bands denoting 21, 22, 23, and 24 cm)
15. Followed by an interval of 9 mm of unmarked thread
16. Followed by 6 coloured bands of 1 mm thickness with intervals of 1 mm of unmarked thread between consecutive bands (the first band at 25 cm, the cluster of second to sixth collectively denoting 25 cm, and the sixth denoting 26 cm)
17. Followed by an interval of 9 mm of unmarked thread
18. Followed by a coloured band of 1 mm thickness, (denoting 27 cm)
19. Followed by 9 mm of unmarked thread.

### 9. INDUSTRIAL APPLICABILITY

There are over 150 million IUS users worldwide. The product will have a corresponding demand, and its production will correspondingly be commercially viable. Product reproducibility is generally very high due to the ease of manufacture.

### Annexure 1

### Specifications as per the 2016 World Health Organization/United Nations Family Planning Association Guideline

### Thread Requirements:

The thread shall be a monofilament made from HDPE, free of stabilizers, with sufficient tensile strength to meet the specified thread breaking force requirement of 9.5 Newton. A minimum tensile strength (ASTM D638-ISO 527-2) of 28 MPa is recommended. The thread polymer shall be compounded with 0.4% up to 1.0% by weight USP (EP) rutile titanium dioxide.

Thread must pass rabbit implantation test after at least 72 hours (histology compared to strips of USP Negative Control Plastic Standard).

The compounded T frame polymer (LDPE plus barium sulphate) and thread or compounded thread polymer shall be evaluated for biological safety in accordance with ISO 10993-1 requirements for mucosal membrane contact devices intended for permanent contact. Specifically, the following evaluations are required:
genotoxicity according to ISO 10993-3
irritation and delayed-type hypersensitivity according to ISO 10993-10;
subacute and subchronic toxicity according to ISO 10993-11 local effects after implantation according to ISO 10993-6; and
cytotoxicity according to ISO 10993-5.

For a specific material it is only necessary to carry out the assessment of biological safety once. The evaluation shall be repeated if there is a significant change to the materials, for example, if the grade or supplier is changed. Manufacturers may continue to use DuPontTM 20 LDPE and Phillips 6007 HDPE without conducting the biocompatibility evaluation.

The product is to be manufactured in a controlled environment so that the bioburden on the products prior to terminal sterilisation can be controlled.
EU—Class III medical devices
USA—combination products.

What is claimed is:

1. A method of using a monofilament thread, consisting of one or more tails, with a fixed, uniform, graded, scale marked on it, made of a material inert to biological fluids, as a retrieval thread for use with intrauterine systems/devices or other intracavitary devices, which are positioned inside the uterus of a woman, or body cavity of a person, comprising the following steps:
a. Providing a manufactured intrauterine system/device or other intracavitary device with the said monofilament thread or retrieval thread;
b. The said monofilament thread to comprise one or more tails of approximately equal length;
c. Marking the said monofilament thread with gradations at fixed uniform intervals with a material inert to biological fluids;
d. Positioning the said device inside the uterus of a woman or body cavity of a person seeking gynecological or relevant intervention.

2. The method of claim 1, wherein the said monofilament thread comprises one or more tails marked with gradations at fixed uniform intervals which are visible to the naked eye and are sono- and radio-opaque.

3. A process of fabricating a monofilament retrieval thread for use with intrauterine systems/devices or other intracavitary devices comprising the following steps:
a. Marking the gradations at fixed uniform intervals on one or more tails of a monofilament thread at about but not limited to 1 cm intervals; the fixed uniform intervals or the gradations/markers can be centimetres, millimetres, or inches, or whole, fractional, or other multiples of these or other units;
b. Marking the gradations at fixed uniform intervals on the tails of a monofilament thread comprising but not limited to 1 cm intervals which has an overall length of about 3600 mm;
c. Marking the gradations at fixed uniform intervals on the tails of a monofilament thread comprising but not limited to 1 cm intervals which has an overall length of about 5400 mm;
d. The process of fabricating the gradation markings at fixed uniform intervals on the monofilament thread using a material stable at a pH range of about 1-9.

4. The process of fabrication of claim 3 wherein the gradations at fixed uniform intervals on the tails of the monofilament threads are marked with a material which is visible to the naked eye and is sono- and radio-opaque.

5. The process of fabrication of claim 3 wherein the gradations at fixed uniform intervals on the tails of the monofilament thread will retain their colour at visual inspection or at speculum examination by a clinician for at least 17 years allowing for the standard shelf life and maximum intended time within the uterine or other body cavity.

6. The process of claim 3 wherein the gradations at fixed uniform intervals on the tails of the monofilament thread can guide a clinician upon inspection to determine whether the intrauterine system or device (or other intracavitary device) has been dislodged or moved from the intended site of placement.

7. A monofilament retrieval thread for use with intrauterine systems/devices or other intracavitary devices prepared using the process of claim 3.

8. A monofilament retrieval thread for use with intrauterine systems/devices or other intracavitary devices comprising one or more tails marked with gradations at fixed uniform intervals which are visible to the naked eye, are sono- and radio-opaque and inert allowing the clinician multiple inspections over at least 12 years to determine the viability of the device in terms of relevant clinical efficacy for a wearer of the device.
